(19) United States
(12) Patent Application Publication
(10) Pub. No.: US 2006/0088483 A1
(43) Pub. Date: Apr. 27, 2006

(54) Kit and method of applying makeup

(76) Inventor: Ludovic Thevenet, Bourg la Reine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/242,900

(22) Filed: Oct. 5, 2005

Related U.S. Application Data
(60) Provisional application No. 60/619,927, filed on Oct. 20, 2004.

(30) Foreign Application Priority Data
Oct. 5, 2004 (FR) 04 10509
Jul. 8, 2005 (WO) PCT/FR05/50558

(51) Int. Cl. A61K 8/19

(52) U.S. Cl. 424/61; 424/64

(57) ABSTRACT

The present invention relates to a kit for applying makeup to a surface such as the skin, the nails, hair, or the lips, said kit comprising: a first cosmetic composition including magnetic particles that are movable under the effect of a magnetic field; a second cosmetic composition for covering or for being covered by the first composition.

### FIELD OF THE INVENTION

The present invention relates to-a kit for applying makeup to a surface, such as the skin, the nails, hair, the lips, or even false nails, and it also relates to a method of applying makeup.

The term “cosmetic composition” as used in the context of the present invention means a composition as defined in the Jun. 14, 1993 EEC Directive 93/35 modifying EEC Directive 76/768. Foundations, lipsticks, and nail varnishes are examples of cosmetic compositions.

### OBJECTS AND SUMMARY OF THE INVENTION

A need exists to benefit from novel appearance effects in the field of makeup and the invention seeks to satisfy that need.

A Kit for Applying Makeup

According to one of its aspects, the invention provides a kit for applying makeup to a surface (S) such as the skin, the nails, hair or the lips, said kit comprising:

a first cosmetic composition (C₁) including metallic iron particles, in particular soft iron; and

a second cosmetic composition (C₂) for covering or for being covered by the first composition (C₁).

According to another of its aspects, the invention provides a kit for applying makeup to a surface such as the skin, the nails, hair, the lips, or even false nails, said kit comprising:

a first cosmetic composition including magnetic particles that are movable under the effect of a magnetic field;

a second cosmetic composition for covering or for being covered by the first composition, said kit may also comprise:

a magnetic device for generating the magnetic field that makes it possible to displace and/or modify the orientation of all or some of the magnetic particles when the first composition is applied in the form of at least one layer to the surface.

The term “movable” means that the orientation and/or the position of the particles can be modified.

The appearance of the first composition deposited on the surface depends on the orientation and/or the position of the magnetic particles. The invention makes it possible to create novel makeup effects, enabling patterns in relief to be produced, for example, or imparting an impression of relief or various other possibly geometrical patterns.

The second composition may be transparent. When the second cosmetic composition is applied to the first, it makes it possible to obtain a depth, gloss, smoothness, or other effect.

The second composition may include a coloring agent, e.g. pigments. When the second composition is colored, it possible to create a colored background/base, for example, the second composition thus being covered by the first, for example.

In particular, the kit of the invention may be used to apply makeup to the lips or to the nails.

The kit of the invention may include a magnetic device for generating a magnetic field that makes it possible to modify the appearance of the first composition, at least immediately after it has been applied to the surface.

First Cosmetic Composition

After a given drying time, the first composition may take on a state that prevents the magnetic particles from further changing their orientation under the effect of a magnetic field. This applies to a nail varnish, for example. In some circumstances, the orientation of the magnetic particles may also be modified at any time, in particular when the first composition does not dry, or presents a very long drying time. This may apply to a foundation, for example.

As mentioned-above, the first composition contains magnetic particles which may be presented in various forms.

Magnetic Particles

The term “magnetic particles”, also termed as “magnetic bodies”, means particles presenting magnetic susceptibility, i.e. particles that are sensitive to the action of a magnetic field, and that tend to come into alignment with the field lines, for example.

The first composition may contain both magnetic particles and non-magnetic particles.

The presence of magnetic particles and of non-magnetic particles in the composition makes it possible to create novel optical effects that can be modulated under the effect of a magnetic field, for example.

In the absence of a magnetic field, the magnetic particles used preferably do-not present any remanent magnetism.

The magnetic particles may comprise any magnetic material that presents sensitivity to the lines of a magnetic field, regardless of whether the field is produced by a permanent magnet or is the result of induction, the material being selected from nickel, cobalt, iron, and alloys and oxides thereof, in particular Fe₃O₄, and also from gadolinium, terbium, dysprosium, erbium, and alloys and oxides thereof, for example. The magnetic material may be of the “soft” or of the “hard”; type.

The magnetic particles may optionally present a multilayer structure including at least one layer of a magnetic material such as iron, nickel, cobalt, and alloys and oxides thereof, in particular Fe₃O4, for example.

The magnetic particles are preferably aspherical, presenting an elongate shape, for example. Thus, when the particles are subjected to the magnetic field, they tend to become oriented with their longitudinal axes in alignment with the field lines, and they are subjected to a change in orientation which results in the first composition changing in appearance.

When the magnetic particles are substantially spherical, their appearance is preferably non-uniform, so that a change in orientation results in a change in appearance.

The quantity of magnetic particles is sufficient to enable the appearance of the composition to depend on their orientation and/or on their positioning.

The concentration of magnetic particles may be in the range about 0.05% to about 97% by weight, for example, preferably in the range about 0.1% to about 95% by weight, and preferably in the range 0.1% to 90% by weight, e.g. about 3% by weight. By way of example, the size of the magnetic particles may be in the range 1 nanometer (nm) to 700 micrometers (pm), e.g. in the range 1 μm to 500 μm, and more preferably in the range about 10 μm to about 150 μm. The term “size” means the size given by the statistical grain size distribution at half the population, referred to as “D50”.

The magnetic particles of the first composition may comprise magnetic pigments. Particularly suitable pigments are nacres comprising iron oxide Fe₃O₄. By way of example, pigments presenting magnetic properties are those sold under the trade names COLORONA BLACKSTAR BLUE, COLORONA BLACKSTAR GREEN, COLORONA BLACKSTAR GOLD, COLORONA BLACKSTAR RED, CLOISONNE NU ANTIQUE SUPER GREEN, MICRONA MATTE BLACK (17437), MICA BLACK (17260), COLORONA PATINA SILVER (17289), and COLORONA PATINA GOLD (117288) by MERCK, or indeed FLAMENCO TWILIGHT RED, FLAMENCO TWILIGHT GREEN, FLAMENCO TWILIGHT GOLD, FLAMENCO TWILIGHT BLUE, TIMICA NU ANTIQUE SILVER 110 AB, TIMICA NU ANTIQUE GOLD 212 GB, TIMICA NU-ANTIQUE COPPER 340 AB, TIMICA NU ANTIQUE BRONZE 240 AB, CLOISONNE NU ANTIQUE GREEN 828 CB, CLOISONNE NU ANTIQUE BLUE 626 CB,. GEMTONE MOONSTONE G 004, CLOISONNE NU ANTIQUE RED 424 CHROMA-LITE, BLACK (4498), CLOISONNE NU ANTIQUE ROUGE FLAMBE (code 440 XB), CLOISONNE NU ANTIQUE BRONZE (240 XB), CLOISONNE NU ANTIQUE GOLD (222 CB), and CLOISONNE NU ANTIQUE COPPER (340 XB) by ENGELHARD.

The magnetic particles may be magnetic fibers.

Magnetic Fibers

The term “fibers” means generally elongate bodies presenting, for example, a form factor in the range 3.5 to 2500 or 5 to 500, e.g. 5 to 150. The form factor is defined by the ratio L/D, where L is the length of the fiber and D is the diameter of the circle in which the widest cross-section of the fiber is inscribed.

By way of example, the cross-section of the fibers may be inscribed in a circle having a diameter in the range 2 nm to 500 μm, e.g. in the range 100 nm to 100 μm, or even 1 μm to 50 μm.

By way of example, the fibers may present a length in the range 1 μm to 10. millimeters (mm), e.g. 0.1 mm to 5 mm, or even 0.3. mm to 3.5 mm.

By way of example, the fibers may present a weight in the range 0.15 denier to 30 denier (weight in grams for 9 km of thread), e.g. 0.18 denier to 18 denier.

The cross-section of the fibers may be of any shape, e.g. circular, or polygonal, in particular square, hexagonal, or octagonal.

The composition may contain solid or hollow fibers that may be independent or interlinked, e.g. braided.

The composition may contain fibers having ends that are blunted and/or rounded, e.g. by polishing.

The shape of the fibers need not be significantly modified when they are inserted into the composition, with said fibers being initially rectilinear and sufficiently rigid to keep their shape. In a variant, the fibers may present flexibility that enables them to be substantially deformed within the composition.

The fibers may contain a non-zero amount, that may be as great as 100%, of a magnetic material selected from soft magnetic materials, hard magnetic materials, in particular based on iron, zinc, nickel, cobalt, or manganese, and alloys and oxides thereof, in particular Fe₃O₄, rare earths, barium sulfate, iron-silicon alloys, possibly containing molybdenum, Cu₂MnAl, MnBi, or a mixture thereof, this list not being limiting.

When the composition contains fibers containing magnetic particles, said magnetic particles may be present at least at the surface of the fibers, or only at the surface of the fibers, or only inside the fibers, or they may even be dispersed within the fibers in substantially uniform manner, for example.

By way of example, each fiber may include a non-magnetic core with a plurality of magnetic particles on its surface.

Each fiber may also include a synthetic matrix containing a plurality of magnetic grains dispersed therein.

Where appropriate, a synthetic material filled with magnetic particles may itself be covered by a non-magnetic membrane. By way of example, such a membrane constitutes a barrier isolating the magnetic material(s) from the surrounding environment and/or it can provide color. Each fiber may comprise a one-piece magnetic core and be covered by a non-magnetic membrane, or it may comprise a one-piece non-magnetic core and be covered by a magnetic membrane.

The composition may contain fibers made by extruding or co-extruding one or more polymeric materials, in particular thermoplastics and/or elastomers. One of the extruded materials may contain a filler of dispersed magnetic particles.

Each fiber may comprise a synthetic material selected from polyamides; polyethylene terephthalate (PET); acetates; polyolefins, in particular polyethylene (PE) or polypropylene (PP); polyvinyl chloride (PVC); polyester block amide; plasticized Rilsan®; elastomers, in particular polyester elastomers, polyethylene (PE) elastomers, silicone elastomers, nitrile elastomers; or a mixture of these materials, this list not being limiting.

The composition may contain composite fibers each comprising a magnetic core that is covered, at least in part, by at least one non-magnetic, synthetic, or natural material. By way of example, the magnetic core may be covered by co-extruding a membrane made of a non-magnetic material around the core.

The core may also be covered in some other way, e.g. by polymerization in situ.

The core may be a single piece or it may include a filler of magnetic grains dispersed in a matrix.

The composition may also contain composite fibers obtained by covering a non-magnetic, synthetic, or natural core, with a synthetic material filled with magnetic particles, the core being composed of a fiber made of wood; rayon; polyamide; plant matter; or polyolefin, in particular polyethylene, Nylon®, polyimide-amide, or aramid, this list not being limiting.

The composition may also contain magnetic composite particles, in particular a magnetic latex.

Magnetic Composite Particles

A magnetic composite particle is a composite material constituted by an organic or an inorganic matrix and by magnetic grains. At their surfaces and/or within themselves, the magnetic composite particles may thus include grains of a magnetic material. The composite particles may be constituted by a magnetic core covered by an organic or an inorganic matrix, or they may be constituted by an organic or an inorganic core covered by a magnetic matrix.

The magnetic-composite particles include one of the above-mentioned magnetic materials, for example.

The size of the magnetic composite particles may be in the range 1 nm to 1 mm, for example, preferably in the range 100 nm to 500 μm, and more preferably in the range 500 nm to 100 μm. The term “size” means the size given by the statistical grain size distribution at half the population, referred to as “D50”.

The thesis by C. GOUBAULT, dated Mar. 23, 2004, and incorporated herein by reference, refers, in chapter 1, to the prior art on the subject of magnetic composite particles, and draws up a list of preparation methods that are suitable for being used to prepare magnetic composite particles, namely separately synthesizing the magnetic grains and the matrix, synthesizing the magnetic grains in contact with the matrix, or synthesizing the matrix in the presence of the magnetic grains.

KISKER markets inorganic-matrix magnetic composite particles composed of silica. DYNAL, SERADYN, ESTAPOR, and ADEMTECH propose organic-matrix magnetic composite particles that are also suitable for being used in the invention.

More particularly, under the reference M1-070/60, ESTAPOR markets magnetic latex constituted by grains of ferrite that are evenly distributed in a polystyrene matrix, said latex including 65% iron oxide, the mean diameter of the polystyrene particles being 890 nm, and the dry material mass content being 10%.

Ferrofluid

The composition may contain a ferrofluid, i.e. a stable colloidal suspension of magnetic particles, in particular of magnetic nanoparticles.

The particles, having a size of the order of several tens of nanometers, for example, are dispersed in a solvent (water, oil, organic solvent), either by means of a surfactant or a dispersant, or by electrostatic interactions.

By way of example, the ferrofluids can be prepared by grinding ferrites or other magnetic particles until nanoparticles are obtained, which particles are then dispersed in a fluid containing a surfactant which is absorbed by the particles and stabilizes them, or else they can be prepared by precipitating a metallic-ion solution in a basic medium.

Each particle of the ferrofluid presents a magnetic moment that is determined by the size of the particle, and by the nature of the magnetic material.

Under the action of a magnetic field, the magnetic moments of the particles tend to come into alignment with the field lines with non-zero magnetization appearing in the liquid. If the field is removed, there is no hysteresis and magnetization drops to zero.

Beyond a field threshold value, it is also possible to cause macroscopic changes in the liquid, e.g. the appearance of peaks, or a change in Theological properties.

The term “ferrofluid” also encompasses an emulsion of ferrofluid droplets in a solvent. Each drop thus contains colloidal magnetic particles in stable suspension. This makes it possible to have a ferrofluid in any type of solvent. The size of the magnetic particles in suspension in the ferrofluid may be in the range 1 nm to 10 μm, for example, preferably in the range 1 nm to 1 μm, and more preferably in the range 1 nm to 100 nm. The term “size” means the size given by the statistical grain size distribution at half the population, referred to as “D50”.

Mention can be made in particular of ferrofluids sold by Liquids Research LTD under the references:

WHKSlS9 (A, B, or C), which is a water-based ferrofluid containing magnetite (Fe₃O₄), having particles of 10 nm in diameter.

WHJS1 (A, B, or C), which is an isoparaffin-based ferrofluid, containing magnetite (Fe₃O₄) particles that are 10 nm in diameter.

BKS25_dextran, which is a water-based ferrofluid stabilized by dextran, containing magnetite (Fe₃O₄) particles that are 9 nm in diameter.

Chains of Particles and/or of Magnetic Fibers

The composition may contain clumps of particles or fibers having a largest dimension, e.g. length, that may, for example, be in the range 1 nm to 10.mm, e.g. in the range 10 nm to 5 mm, or in the range 100 nm to 1 mm, or even in the range 0.5 μm to 3.5 mm, e.g. in the range 1 μm to 150 μm. The term “size” means the size given by the statistical grain size distribution at half the population, referred to as “D50”.

By way of example, chains of magnetic particles may be obtained by assembling colloidal magnetic particles, as described in the publications “Permanently linked monodisperse paramagnetic chains”, by E. M. Furst, C. Suzuki, M. Fermigier, A. P. Gast, Langmuir, 14, 7334-7336 (1998), “Suspensions of magnetic particles”, by M. Fermigier, Y. Grasselli, Bulletin of the SFP (105) July 1996, and “Flexible magnetic filaments as micromechanical sensors”, by C. Goubault, P. Jop, M. Fermigier, J. Baudry, E. Bertrand, J. Bibette, Phys. Rev. Lett., 91, 26, 260802-1 to 260802-4 (2003), the contents of which are incorporated herein by reference.

In particular, those articles describe how to proceed in order to obtain chains of magnetic-latex particles that include a polystyrene matrix containing grains of iron oxide with functions on the surface, and that are bonded together in permanent manner following a chemical reaction, in particular covalent bonds between the surfaces of adjacent particles; a method is also described of obtaining chains of ferrofluid-emulsion droplets that are bonded together by physical interactions. The length and the diameter of the permanent chains obtained in this way can be controlled. Such magnetic chains constitute anisotropic magnetic objects that can be oriented and displaced under the effect of a magnetic field.

The dimensions of the magnetic chains may satisfy the same conditions as for the magnetic fibers.

By way of example, the first composition may contain at least one goniochromatic coloring agent in which a color change can be observed as a function of the angle of observation. The goniochromatic coloring agent may optionally be magnetic.

When the first composition contains magnetic particles of a certain color and a goniochromatic coloring agent, said coloring agent may be selected so that its range of colors passes substantially through the color of the magnetic particles.

By way of example, this can make the magnetic particles more difficult to detect so long as they are not oriented under the effect of a magnetic field.

This can also allow the pattern induced by orienting the magnetic particles to appear clearly only when the made-up surface is under certain observation and/or lighting conditions.

Goniochromatic Coloring Agents

The term “goniochromatic coloring agent” as used in the context of the present invention means a coloring agent that makes it possible, when the composition is spread on a surface, to obtain a color path in the a*b* plane of the 1976 CIE color space which corresponds to a variation Dh of the tint angle h of at least 20° when the angle of observation is varied relative to the normal in the range 0° to 80° for light at an angle of incidence of 45°.

By way of example, the color path may be measured by means of a spectrogonioreflectometer, from INSTRUMENT SYSTEMS and referenced GON 360 GONIOMETER, after the first composition has been spread in the fluid state to a thickness of 300 μm by means of an automatic spreader on a contrast card from ERICHSEN and referenced Typ 24/5, the measurements being performed on the black background of the card.

By way of example, the goniochromatic coloring agent may be selected from multilayer interference structures and liquid crystal coloring agents.

By way of example, a multilayer structure may comprise at least two layers, each layer being produced, for example, from at least one material selected from the group constituted by the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers, and combinations thereof.

The multilayer structure may optionally be symmetrical with respect to a central layer as regards the chemical nature of the stacked layers. Depending on the thickness and nature of the various layers, different effects are obtained.

Examples of symmetrical multilayer interference structures are as follows: Fe₂O₃/SiO₂/Fe₂O₃/SiO₂/Fe₂O₃, a pigment having this structure being sold under the trade name SICOPEARL by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂, pigments with these structures being sold under the trade name XIRONA by MERCK (Darmstadt).

By way of example, liquid crystal coloring agents comprise silicones, or cellulose ethers onto which mesomorphic groups have been grafted. Examples of suitable liquid crystal goniochromatic particles are those sold by CHENIX, and those sold under the trade name HELICONE® HC by WACKER.

Suitable goniochromatic coloring agents are some nacres; pigments having effects on synthetic substrates, in particular alumina, silica, borosilicate, iron oxide, or aluminum type substrates; or holographic interference flakes coming from a polyterephthalate film.

The material may further contain dispersed goniochromatic fibers. Such fibers could present a length that is less than 80 μm, for example.

The first composition may also contain at least one diffractive pigment which may present magnetic properties if necessary.

Diffractive Pigments

The term “diffractive pigment” as used in the context of the present invention means a pigment that is capable of producing a variation in color depending on the angle of observation when lit by white light, because of the presence of a structure which diffracts the light.

A diffractive pigment may include a diffraction grating that is capable of diffracting an incident ray of monochromatic light in defined directions.

The diffraction grating may comprise a periodic pattern, in particular a line, with the distance between two adjacent patterns being the same as the wavelength of the incident light.

When the incident light is polychromatic, the diffraction grating separates the various spectral components of the light and produces a rainbow effect.

With regard to the structure of diffractive pigments, reference can usefully be made to the article “Pigments Exhibiting Diffractive Effects” by Alberto Argoitia and Matt Witzman, 2002, Society of Vacuum coaters, 45^{th} Annual Technical Conference Proceedings 2002.

The diffractive pigment may be made with patterns having various profiles, in particular triangular, optionally symmetrical, notched, of optionally constant width, or sinusoidal.

The spatial frequency of the grating and the depth of the patterns are selected as a function of the degree of separation of the various desired orders. The frequency may be in the range 500 lines per mm to 3000 lines per mm, for example.

Each of the particles of the diffractive pigment preferably presents a flat shape, and in particular a wafer shape.

A single pigment particle may include two crossed diffraction gratings that are optionally perpendicular.

The diffractive pigment may present a multilayer structure comprising a layer of reflective material that is covered on at least one side by a layer of dielectric material. The dielectric material may make the diffractive pigment stiffer and longer lasting. For example, the dielectric material may thus be selected from the following materials: MgF₂, SiO₂, Al₂O₃, AlF₃, CeF₃, LaF₃, NdF₃, SmF₂, BaF₂, CaF₂, LiF, and combinations thereof. For example, the reflective material may be selected from metals and alloys thereof, and also from non-metallic reflective materials. Metals that may be used include Al, Ag, Cu, Au, Pt, Sn, Ti, Pd, Ni, Co, Rd, Nb, Cr, and materials, combinations, or alloys thereof. Such a reflective material may, on its own, constitute the diffractive pigment which then comprises a single layer.

In a variant, the diffractive pigment may include a multilayer structure comprising a core of dielectric material with a reflective layer covering at least one side, or indeed completely encapsulating, the core. A layer of dielectric material may also cover the reflective layer(s). The dielectric material used is thus preferably inorganic, and may, for example, be selected from metal fluorides, metal oxides, metal sulfides, metal nitrides, metal carbides, and combinations thereof. The dielectric material may be in the crystalline, semi-crystalline, or amorphous state. In this configuration, the dielectric material may, for example, be selected from the following materials: MgF₂, SiO, SiO₂, Al₂O₃, TiO₂, WO, AlN, BN, B₄C, WC, TiC, TiN, N₄Si₃, ZnS, glass particles, diamond-type carbons, and combinations thereof.

In a variant, the diffractive pigment may be composed of a preformed dielectric or ceramic material such as a naturally lamellar mineral, e.g. mica peroskovite or talc; or synthetic platelets formed from glass, alumina, SiO₂, carbon, an iron oxide/mica, mica covered in BN, BC, graphite, or bismuth oxychloride, and combinations thereof.

Instead of a layer of dielectric material, other materials that improve the mechanical properties may be suitable. Such materials may include silicone, metal silicides, semiconductor materials formed from elements of groups III, IV, and V, metals having a body centered cubic crystal structure, metal-ceramic compositions or materials, semiconductor glasses, and various combinations thereof.

In particular, the diffractive pigment used may be selected from those described in US patent application No. 2003/0031870 published on Feb. 13, 2003.

A diffractive pigment may, for example, have the following structure: MgF₂/Al/MgF₂, a diffractive pigment having this structure being sold by FLEX PRODUCTS under the trade names SPECTRAFLAIR 1400 Pigment Silver or SPECTRAFLAIR 1400 Pigment Silver FG. The proportion by weight of MgF₂ may be in the range 80% to 95% of the total weight of the pigment.

By way of example, the quantity of diffractive pigment may be in the range 0.1% to 5% by weight relative to the total weight of the first composition.

By way of example, the size of the diffractive pigment may be in the range 5 μm to 200 μm, and preferably in the range 5 μm to 100 μm, e.g. in the range 5 μm to 30 μm.

The thickness of the diffractive-pigment particles may be less than or equal to 3 μm, or preferably 2 μm, e.g. about 1 μm.

Reflective Particles

By way of example, the first composition may contain reflective particles, in particular optionally-magnetic flakes, amongst others.

The term “reflective particles” as used in the context of the present invention means particles the size and structure of which, in particular the thickness of the layer or layers constituting them and their physical and chemical natures, and their surface state, allow them to reflect incident light. If appropriate, said reflection may have sufficient intensity to create highlight points on the surface of the composition or of the mixture, when the composition or the mixture is applied to the surface to be made up, which highlight points are visible to the naked eye, i.e. they are points of greater brightness that contrast with their environment and appear to shine.

The reflective particles may be selected in a manner such that they do not significantly alter the coloring effect generated by the coloring agents associated therewith, and more particularly to optimize that effect in terms of color yield. More particularly, they may have a yellow, pink, red, bronze, orangey, brown, and/or copper glint.

The reflective particles may be present in the first composition in an amount in the range 0.5% to 60% by weight relative to the total weight of the first composition, specifically 1% to 30% by weight, and in particular 3% to 10% by weight.

Said particles may be in various forms, in particular they may be in the form of flakes, or they may be globular, in particular spherical.

Regardless of their form, the reflective particles may optionally have a multilayer structure; with a multilayer structure, for example, they may have at least one layer of uniform thickness, in particular of a reflective material.

When the reflective particles do not have a multilayer structure, they may, for example, be composed of metal oxides, in particular oxides of titanium or iron obtained by synthesis.

When the reflective particles have a multilayer structure they may, for example, comprise a natural or synthetic substrate, in particular a synthetic substrate which is at least partially coated with at least one layer of a reflective material, in particular at least one metal or metallic material. The substrate may be a single material or multiple materials, and it may be organic and/or inorganic.

More particularly, it may be selected from glasses, ceramics, graphite, metal oxides, aluminas, silicas, silicates, in particular aluminosilicates and borosilicates, synthetic mica, and mixtures thereof, this list not being limiting.

The reflective material may comprise a layer of metal or of a metallic material.

Reflective particles have been described in particular in Japanese patent documents JP-A-09188830, JP-A-10158450, JP-A-10158541, JP-A-07258460 and JP-A-05017710.

Further examples of reflective particles comprising a mineral substrate coated with a metal layer that may be mentioned are particles comprising a substrate of borosilicate coated with silver, also termed “white nacres”.

Glass substrate particles coated with silver in the form of flakes are sold under the trade name MICROGLASS METASHINE REFSX 2025 PS by TOYAL. Glass substrate particles coated with nickel/chromium/molybdenum alloy are sold under the trade name CRYSTAL STAR GF 550, GF 2525 by the same company.

Reflective particles of any form may also be selected from particles of synthetic substrate at least partially coated with at least one layer of at least one metallic material, in particular a metal oxide selected, for example, from oxides of titanium, in particular TiO₂, of iron, in particular Fe₂O₃, of tin, or of chromium, barium sulfate, and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and their mixtures or alloys.

Examples of such particles that may be mentioned are particles comprising a substrate of synthetic mica coated with titanium dioxide, or glass particles coated either with brown iron oxide, titanium oxide, tin oxide, or one of their mixtures such as those sold under the trade name REFLECKS® by ENGELHARD.

The first composition of the invention may contain at least one optionally-magnetic nacre.

Nacres

The term “nacre” means colored particles of any form, which may optionally be iridescent, as produced in the shells of certain mollusks, or which are synthesized, and which exhibit a “pearlescent” coloring effect by optical interference.

Nacres may be selected from nacre pigments such as mica titanium coated with iron oxide, mica coated with bismuth oxychloride, mica titanium coated with chromium oxide, mica titanium coated with an organic colorant, in particular of the type mentioned above, and nacre pigments based on bismuth oxychloride. They may also be particles of mica on the surface of which at least two successive layers of metal oxides and/or organic coloring substances have been superimposed.

More particularly, the nacres may have a yellow, pink, red, bronze, orangey, brown, gold, and/or coppery color or glint.

Illustrative examples of nacres suitable for being introduced into the first composition and that may be mentioned are gold color nacres, in particular those sold by ENGELHARD under the trade names Brillant gold 212G (Timica), Gold 222C (Cloisonne), Sparkle gold (Timica), Gold 4504 (Chromalite), and Monarch gold 233X (Cloisonne); bronze nacres, in particular those sold by MERCK under the trade names Bronze fine (17384) (Colorona) and Bronze (17353) (Colorona), and by ENGELHARD under the trade name Super bronze (Cloisonne); orange nacres especially those sold by ENGELHARD under the trade names Orange 363C (Cloisonne) and Orange MCR 101 (Cosmica), and by MERCK under the trade names Passion orange (Colorona) and Matte orange (17449) (Microna); brown-tinted nacres sold by ENGELHARD under the trade names Nu-antique copper 340XB (Cloisonne) and Brown CL4509 (Chromalite); nacres with a copper glint sold by ENGELHARD under the trade name Copper 340A (Timica); nacres with a red glint, especially those sold by MERCK under the trade name Sienna fine (17386) (Colorona); nacres with a yellow glint, especially those sold by ENGELHARD under the trade name Yellow (4502) (Chromalite); red-tinted nacres with gold glints, especially those sold by ENGELHARD under the trade name Sunstone G012 (Gemtone); pink nacres, especially those sold by ENGELHARD under the trade name Tan opale G005 (Gemtone); black nacres with a glint, especially those sold by ENGELHARD under the trade name Nu antique bronze 240 AB (Timica); blue nacres, especially those sold by MERCK under the trade name Matte blue (17433) (Microna); white nacres with silvery glints, especially those sold by MERCK under the trade name Xirona Silver; and orange-pink green-gold highlight nacres sold by MERCK under the trade names Indian summer (Xirona) and mixtures thereof.

By way of example, the first composition may contain at least one optionally-magnetic filler.

Fillers

The term “filler” means particles of any form which are insoluble in the composition medium regardless of the temperature at which the composition is manufactured. A filler primarily acts to modify the rheology or texture of the composition. The nature and quantity of the particles could depend on the desired mechanical properties and textures.

Examples of fillers that may be mentioned include amongst others talc, mica, silica, kaolin, and sericite, and powders of polyamide, polyolefin, e.g. polyethylene, polytetrafluoroethylene, polymethylmethacrylate, or polyurethane, powdered starch, and silicone resin beads.

Amongst other things, the fillers may be intended to create a fuzzy effect, in particular for a foundation, so as to conceal skin imperfections.

The first composition may also contain colorants, organic pigments, or lakes.

Colorants, Organic Pigments, and Lakes

The colorants may be liposoluble or hydrosoluble.

Examples of liposoluble colorants are Sudan red, D&C Red No.17, D&C Green No.6, β-carotene, soybean oil, Sudan brown, D&C Yellow No.11, D&C Violet No.2, D&C orange No.5, and quinoline yellow.

Examples of hydrosoluble colorants are beetroot juice and methylene blue.

By way of example, the colorants may represent 0.1% to 20% by weight of the first or second composition, or even 0.1% to 6%, when present.

The lakes or organic pigments may be selected from the following materials and mixtures thereof:

cochineal carmine;

the organic pigments of azo, anthraquinone, indigo, xanthene, pyrene, quinoline, triphenylmethane, or fluorane dyes;

organic lakes or insoluble salts of sodium, potassium, calcium, barium, aluminum, zirconium, strontium, titanium, or of acid dyes such as azo, anthraquinone, indigo, xanthene, pyrene, quinoline, triphenylmethane, or fluorine dyes, which dyes may comprise at least one carboxylic or sulfonic acid group.

Organic pigments that may be mentioned include those with the following denominations: D&C Blue No.4, D&C Brown No.1, D&C Green No.5, D&C Green No.6, D&C Orange No.4, D&C Orange No.5, D&C Orange No.10, D&C Orange No.11, D&C Red No.6, D&C Red No.7, D&C Red No.17, D&C Red No.21, D&C Red No.22, D&C Red No.27, D&C Red No.28, D&C Red No.30, D&C Red No.31, D&C Red No.33, D&C Red No.34, D&C Red No.36, D&C Violet No.2, D&C Yellow No.7, D&C Yellow No.8, D&C Yellow No.10, D&C Yellow No.11, FD&C Blue No.1, FD&C Green No.3, FD&C Red No.40, FD&C Yellow No.5, FD&C Yellow No.6.

The organic coloring substance may comprise an organic lake supported by an organic support such as colophane or aluminum benzoate, for example.

Particular organic lakes that may be mentioned include those with the following denominations: D&C Red No.2 Aluminum lake, D&C Red No.3 Aluminum lake, D&C Red No.4 Aluminum lake, D&C Red No.6 Aluminum lake, D&C Red No.6 Barium lake, D&C Red No.6 Barium/Strontium lake, D&C Red No.6 Strontium lake, D&C Red No.6 Potassium lake, D&C Red No.7 Aluminum lake, D&C Red No.7 Barium lake, D&C Red No.7 Calcium lake, D&C Red No.7 Calcium/Strontium lake, D&C Red No.7 Zirconium lake, D&C Red No.8 Sodium lake, D&C Red No.9 Aluminum lake, D&C Red No.9 Barium lake, D&C Red No.9 Barium/Strontium lake, D&C Red No.9 Zirconium lake, D&C Red No.10 Sodium lake, D&C Red No.19 Aluminum lake, D&C Red No.19 Barium lake, D&C Red No.19 Zirconium lake, D&C Red No.21 Aluminum lake, D&C Red No.21 Zirconium lake, D&C Red No.22 Aluminum lake, D&C Red No.27 Aluminum lake, D&C Red No.27

Aluminum/Titanium/Zirconium lake, D&C Red No.27 Barium lake, D&C Red No.27 Calcium lake, D&C Red No.27 Zirconium lake, D&C Red No.28 Aluminum lake, D&C Red No.30 lake, D&C Red No.31 Calcium lake, D&C Red No.33 Aluminum lake, D&C Red No.34 Calcium lake, D&C Red No.36 lake, D&C Red No.40 Aluminum lake, D&C Blue No.1 Aluminum lake, D&C Green No.3 Aluminum lake, D&C Orange No.4 Aluminum lake, D&C Orange No.5 Aluminum lake, D&C Orange No.5 Zirconium lake, D&C Orange No.10 Aluminum lake, D&C Orange No.17 Barium lake, D&C Yellow No.5 Aluminum lake, D&C Yellow No.5 Zirconium lake, D&C Yellow No.6 Aluminum lake, D&C Yellow No.7 Zirconium lake,. D&C Yellow No.10 Aluminum lake, FD&C Blue No.1 Aluminum lake, FD&C Red No.4 Aluminum lake, FD&C Red No.40 Aluminum lake, FD&C Yellow No.5 Aluminum lake, FD&C Yellow No.6 Aluminum lake.

The chemical materials corresponding to each of the organic coloring substances listed above are mentioned in the work entitled “International Cosmetic Ingredient Dictionary and Handbook”, 1997 edition, pages 371 to 386 and 524 to 528, published by “The Cosmetic, Toiletry, and Fragrance Association”, the contents of which are incorporated herein by reference.

The first composition may contain a composite pigment.

Composite Pigments

The composite pigment may be composed of particles comprising:

an optionally-magnetic inorganic core; and

at least one at least partial coating of at least one organic coloring substance.

At least one binder may advantageously contribute to fixing the organic coloring substance onto the inorganic core.

The particles of composite pigment may have a variety of forms. In particular, said particles may be in the form of flakes or they may be globular, in particular spherical, and may be hollow or solid. The term “in the form of flakes” means particles for which the ratio of the largest dimension to the thickness is 5 or more.

A composite pigment may, for example, have a specific surface area in the range 1 square meter per gram (m²/g) to 1000 m²/g, in particular in the range about 10 m²/g to about 600 m²/g, and in particular in the range about 20 m²/g to about 400 m²/g. The specific surface area is the value measured using the BET (Brunauer-Emmett-Teller) method.

The inorganic core of the composite pigment may have any form that is suitable for fixing particles of organic coloring substance, for example spherical, globular, granular, polyhedral, acicular, spindle-shaped, flattened in the form of a flake, a rice grain, or a scale, and a combination of these forms, this list not being limiting.

The ratio of the largest dimension of the core to its smallest dimension may be in the range 1 to 50.

The inorganic core may have a size in the range about 1 nm to about 100 nm, or even in the range about 5 nm to about 75 nm, for example in the range about 10 nm to about 50 nm.

The inorganic core may be formed from a material selected from a non-limiting list comprising metallic salts and metal oxides, in particular oxides of titanium, zirconium, cerium, zinc, iron, iron blue, aluminum, and chromium, aluminas, glasses, ceramics, graphite, silicas, silicates, in particular aluminosilicates and borosilicates, synthetic mica, and mixtures thereof.

Oxides of titanium, in particular TiO₂, of iron, especially Fe₂O₃, of cerium, zinc, and aluminum, silicates, in particular aluminosilicates and borosilicates, are particularly suitable.

The inorganic core may have a specific surface area, measured using the BET method, in the range about 1 m²/g to about 1000 m²/g, preferably in the range about 10 m²/g to about 600 m²/g, for example in the range about 20 m²/g to about 400 m²/g.

The inorganic core may be colored if appropriate.

The organic coloring substance may be as defined above.

The composite-pigment binder may be of any type provided that it allows the organic coloring substance to adhere to the surface of the inorganic core.

In particular, the binder may be selected from the following non-limiting list: silicone materials, polymeric, oligomeric or similar materials, in particular from organosilanes, fluoroalkylated organosilanes and polysiloxanes, for example polymethylhydrogen siloxane, as well as a variety of coupling agents such as coupling agents based on silanes, titanates, aluminates, zirconates, and mixtures thereof.

The coloring agent may contain a photochrome coloring substance or a photochrome agent.

Photochrome Agents

In general, a photochrome coloring agent is a coloring agent having the property of changing tint when it is lit by ultraviolet light, and of returning to its initial color when it is no longer lit by said light, or even of passing from a non-colored state to a colored state and vice-versa. In other words, such an agent presents different tints depending on whether it is lit by artificial light, or by light containing a certain amount of UV radiation such as sunlight.

Referende could usefully be made to the examples of photochrome agents described in EP 1 410 786.

Thermochrome Agents

By way of example, it is possible to use the thermochrome agent, reference KROMAFAST YELLOW 5GX 02, sold by KROMACHEM LTD.

Other Coloring Agents

The first composition may also contain piezochrome compounds, in particular tribochromes or solvatochromes.

Other Components

Typically, the cosmetic composition includes a physiologically acceptable medium. The term “physiologically acceptable medium” means a non-toxic medium that can be applied to the skin, to the nails, to hair, or to the lips of human beings. The physiologically acceptable medium is generally adapted to the nature of the surface onto which the composition is to be applied, and to the form in which the composition is packaged.

The first composition may include ingredients other than those described above, in particular at least one solvent, one oily phase, one--film-forming polymer, and/or one dermatologically or cosmetically active ingredient, in particular as a function of its dosage or “galenical” form.

Solvents

The first composition may include at least one aqueous or organic solvent, in particular an organic solvent, e.g. a volatile oil.

The term “volatile solvent” as used in the context of the present invention means a solvent that is liquid at ambient temperature, having a non-zero vapor pressure at ambient temperature and atmospheric pressure, in particular a vapor pressure in the range 0.13 pascals (Pa) to 40000 Pa (10⁻³ millimeters of mercury (mm Hg) to 300 mm Hg), and preferably in the range 1.3 Pa to 13000 Pa (0.01 mm Hg to 100 mm Hg), and preferably in the range 1.3 Pa to 1300 Pa (0.01 mm Hg to 10 mm Hg).

When the first composition contains one or more organic solvents, the solvents may be present in an amount in the range 0.1% to 99%, relative to the total weight of the composition under consideration.

In general, the quantity of solvent(s), in particular organic solvent(s), depends on the nature of the surface to which the composition is intended to be applied.

The first composition may include at least one volatile solvent constituted by a volatile oil.

The oil may be a siliconized oil or a hydrocarbonated oil, or may include a mixture of such oils.

The term “siliconized oil” as used in the context of the present invention means an oil including at least one silicon atom, and in particular at least one Si-O group.

The term “hydrocarbonated oil” means an oil containing mainly hydrogen and carbon atoms and possibly oxygen, nitrogen, sulfur, and/or phosphorus atoms.

The volatile hydrocarbonated oils may be selected from hydrocarbonated oils having 8 to 16 carbon atoms, and in particular C8-C16 branched alkanes (also termed isoparaffins) such as isododecane (also termed 2,2,4,4,6-pentamethylheptane), isodecane, isohexadecane, and oils sold under the trade names Isopars® or Permethyls®, for example.

Volatile oils that may also be used are volatile silicones, such as volatile linear or cyclic silicone oils, for example, in particular oils having a viscosity ≧8 centistokes (cSt) (8×10⁻⁶ square meters per second (m²/s)), and having in particular 2 to 10 silicon atoms, and in particular 2 to 7 silicon atoms, the silicones possibly including alkyl or alkoxy groups having 1 to 10 carbon atoms. In the invention, suitable volatile silicone oils that may be mentioned are in particular dimethicones having a viscosity of 5 cSt to 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

It is also possible to use fluorinated volatile oils such as nonafluoromethoxybutane or perfluoromethylcyclopentane, and mixtures thereof.

The composition may contain 0.01% to 95% by weight of volatile oil relative to the total weight of the composition, and preferably 1% to 75% by weight.

The first composition may comprise at least one organic solvent selected from the following list:

ketones that are liquid at ambient temperature, such as methylethylketone, methylisobutylketone, diisobutylketone, isophorone, cyclohexanone, or acetone;

alcohols that are liquid at ambient temperature, such as ethanol, isopropanol, diacetone alcohol, 2-butoxyethanol, or cyclohexanol;

glycols that are liquid at ambient temperature, such as ethylene glycol, propylene glycol, pentylene glycol, or glycerol;

propylene glycol ethers that are liquid at ambient temperature, such as propylene glycol monomethyl ether, the acetate of propylene glycol monomethyl ether, or dipropylene glycol mono n-butyl ether;

short-chain esters (containing a total of 3 to 8 carbon atoms), such as ethyl acetate, methyl acetate, propyl acetate, n-butyl acetate, or isopentyl acetate; and

alkanes that are liquid at ambient temperature, such as decane, heptane, dodecane, or cyclohexane.

The first composition may also comprise water or a mixture of water and hydrophilic organic solvents which are routinely used in cosmetics, such as alcohols, in particular linear or branched lower monoalcohols containing 2 to 5 carbon atoms, such as ethanol, isopropanol or n-propanol, polyols such as glycerine, diglycerine, propylene glycol, sorbitol, penthylene glycol, or polyethylene glycols. The first composition may also contain hydrophilic C₂ ethers and C₂-C₄ aldehydes. The water or mixture of water and hydrophilic organic solvents may be present in the first and/or second composition in an amount in the range 0% to 90%, in particular 0.1% to 90% by weight, and preferably 0% to 60% by weight, more particularly 0.1% to 60% by weight relative to the total weight of the composition.

Oily Phase

When it is to be applied to the lips, the first composition may, for example, include an oily phase and in particular at least one fat that is liquid at ambient temperature (25° C.) and/or a fat that is solid at ambient temperature, such as waxes, pasty fats, gums, and mixtures thereof. The oily phase may also contain lipophilic organic solvents.

By way of example, the first composition may have a continuous oily phase which may contain less than 5% water, in particular less than 1% water relative to its total weight, and in particular it may be in the anhydrous form.

Fats that are liquid at ambient temperature, usually termed “oils”, that may be mentioned are: hydrocarbon-containing vegetable oils such as liquid fatty acid triglycerides containing 4 to 10 carbon atoms, for example heptanoic or octanoic acid triglycerides, or sunflower, corn, soybean, grapeseed, sesame seed, apricot kernel, macadamia nut, castor, or avocado stone oil, caprylic/capric acid triglycerides, jojoba oil, shea nut butter oil, lanolin, acetylated lanolin; linear or branched hydrocarbons of mineral or synthetic origin, such as paraffin oils and their derivatives, Vaseline, polydecenes, hydrogenated polyisobutene such as Parleam; synthesized esters and ethers, in particular fatty acids such as Purcellin oil, isopropyl myristate, 2-ethylhexyl palmitate, 2-octyldodecyl stearate, 2-octyldodecyl erucate, isostearyl isostearate; hydroxylated esters such as isostearyl lactate, octylhydroxystearate, octyldodecyl hydroxystearate, diisostearylmalate, triisocetyl citrate, fatty alcohol heptanoates, octanoates, or decanoates; isononyl isonanoate, isopropyl lanolate, tridecyl trimellilate, diisostearyl malate; polyol esters such as propylene glycol dioctanoate, neopentylglycol diheptanoate, diethyleneglycol diisononanoate; and pentaerythritol esters; fatty alcohols containing 12 to 26 carbon atoms, such as octyldodecanol, 2-butyloctanol, 2-hexyldecanol, 2-undecylpentadecanol, or oleic alcohol; partially hydrocarbonated and/or siliconized fluorinated oils; silicone oils such as volatile or non-volatile, linear or cyclic polymethylsiloxanes (PDMS) which may be liquid or pasty at ambient temperature, such as cyclomethicones or dimethicones, optionally comprising a phenyl group, such as phenyl trimethicones, phenyltrimethylsiloxydiphenyl siloxanes, diphenylmethyldimethyltrisiloxanes, diphenyl dimethicones, phenyl dimethicones, polymethylphenylsiloxanes; and mixtures thereof. The oils may be present in an amount in the range 0.01% to 90%, and preferably 0.1% to 85% by weight relative to the total weight of the composition.

The presence of an oily phase may impart a gloss effect, and may present a refractive index in the range 1.47 to 1.51, for example, and preferably in the range 1.48 to 1.50. The refractive index is measured at ambient temperature (25° C.) by means of a refractometer.

The first composition may comprise a pasty fat, a wax, or a gum.

Pasty fats are generally hydrocarbon-containing compounds with a melting point in the range 25° C. to 60.5, preferably in the range 30° C. to 45° C., and/or with hardness in the range 0.00-1 megapascals (MPa) to 0.4 MPa, preferably in the range 0.005 MPa to 0.4 MPa, such as lanolins and derivatives thereof.

Waxes may be solid at ambient temperature (25° C.) with a reversible solid/liquid change of state, with a melting point of more than 30.C and up to 200° C., a hardness of more than 0.5 MPa, and with an anisotropic crystalline organization in the solid state. In particular, the waxes may have a melting point of more than 25° C., and preferably more than 45° C. The waxes may be hydrocarbon-containing, fluorinated and/or siliconized and may be of animal, mineral, vegetable and/or synthetic origin. Suitable waxes that may be mentioned are beeswax, carnauba wax or candellila wax, paraffin, microcrystalline waxes, ceresin, or ozokerite; synthetic waxes such as polyethylene or Fischer-Tropsch waxes or silicone waxes such as alkyl or alkoxy-dimethicone containing 16 to 45 carbon atoms. The composition may contain 0 to 50% by weight of waxes relative to the total weight of the composition, or even 1% to 30% by weight.

Suitable gums are generally high molecular weight polydimethylsiloxanes (PDMS) or cellulose gums or polysaccharides.

Film-Forming Polymers

By way of example, the first composition may also include a film-forming polymer, in particular for a mascara or a nail polish. The term “film-forming polymer” means a polymer that can form, by itself or in the presence of an additional film-forming agent, a continuous film that adheres to a surface, in particular to keratinous materials.

Suitable film-forming polymers for use in the first composition in accordance with the invention that may be mentioned include synthetic polymers, of the radical or polycondensate type, natural polymers such as nitrocellulose or cellulose esters, and mixtures thereof.

Radical type film-forming polymers may in particular be vinyl polymers or copolymers, in particular acrylic polymers.

Vinyl film-forming polymers may result from polymerizing monomers with an ethylenically unsaturated bond containing at least one acid group and/or esters of said acid monomers and/or amides of said acid monomers, such as α,β-ethylenically unsaturated carboxylic acids, for example acrylic acid, methacrylic acid, crotonic acid, maleic acid, or itaconic acid.

Vinyl film-forming polymers may also result from homopolymerizing or copolymerizing monomers selected from vinyl esters such as vinyl acetate, vinyl neodecanoate, vinyl pivalate, vinyl benzoate, and vinyl t-butyl benzoate, and styrene monomers such as styrene and alpha-methyl styrene.

Examples of film-forming polycondensates that may be mentioned include polyurethanes, polyesters, polyester amides, polyamides, and polyureas, this list not being limiting.

Polymers of natural origin, which may optionally be modified, may be selected from shellac resin, gum sandarac, dammar resin, gum elemi, copal resin, cellulose polymers such as nitrocellulose, ethylcellulose, or nitrocellulose esters selected, for example, from cellulose acetate, cellulose acetobutyrate, and cellulose acetopropionate, and mixtures thereof.

The film-forming polymer may be present in the form of solid particles in an aqueous or oily dispersion, generally known as latexes or psuedolatexes. The film-forming polymer may comprise one or more stable dispersions of generally spherical polymer particles of one or more polymers in a physiologically acceptable liquid oily phase. Said dispersions are generally termed polymer NADs (non-aqueous dispersions), in contrast to latexes which are aqueous polymer dispersions. Said dispersions may be in the form of nanoparticles of polymers in stable dispersion in said oily phase. The nanoparticle size is preferably in the range 5 nm to 600 nm. Techniques for preparing said dispersions are well known to the person skilled in the art.

Aqueous film-forming polymer dispersions which may be used are acrylic dispersions sold under the trade names NEOCRYL XK-90®, NEOCRYL A-1070®, NEOCRYL A-1090®, NEOCRYL BT-62®′, NEOCRYL A-1079®, NEOCRYL A-523® by AVECIA-NEORESINS, and DOW LATEX 432® by DOW CHEMICAL; DAITOSOL 5000 AD® by DAITO KASEI KOGYO; or aqueous polyurethane dispersions sold under the trade names NEOREZ R-981® and NEOREZ R-974® by AVECIA-NEORESINS; AVALURE UR-405®, AVALURE UR-410®, AVALURE UR-425®, AVALURE UR-450®, SANCURE 875®, SANCURE 861®, SANCURE 878®, and SANCURE 2060® by GOODRICH; IMPRANIL 85® by BAYER; AQUAMERE H-1511® by HYDROMER; and sulfopolyesters sold under the trade mark Eastman AQ by Eastman Chemical Products.

The. first composition of the invention may also comprise an auxiliary film-forming agent which encourages the formation of a film with the film-forming polymer.

Active Ingredients

The first composition may include at least one cosmetically or dermatologically active ingredient. Suitable cosmetically, dermatologically, hygienically, or pharmaceutically active ingredients for use in the compositions of the invention that may be mentioned are moisturizing agents (polyols such as glycerine), vitamins (C, A, E, F, B, or PP), essential fatty acids, essential oils, ceramides, sphingolipids, liposoluble or nanoparticle sun screens, and specific skin treatment active ingredients (protective agents, antibacterials, anti-wrinkle agents, etc), self-tanning agents. Said active ingredients may be used in concentrations in the range 0 to 20%, for example, in particular in the range 0.001% to 15% relative to the total weight of the composition.

The first composition may also contain ingredients that are routinely used in cosmetics, such as thickeners, surfactants, oligo-elements, moisturizing agents, softeners, sequestrating agents, fragrances, alkalinizing or acidifying agents, preservatives, antioxidants, UV filters, colorants, or mixtures thereof.

Depending on the envisaged application, the first composition of the invention may include constituents which are conventionally used in the fields under consideration, and which are present in quantities appropriate to the desired dosage form.

Dosage Forms

The first composition may be in a variety of forms, depending on its purpose. The first composition may thus be in any dosage form that is normally used for topical application, in particular in the anhydrous form, in the form of an oily or aqueous solution, an oily or aqueous gel, an oil-in-water or a water-in-oil emulsion, a wax-in-water or a water-in-wax emulsion, a multiple emulsion or a dispersion of oil in water due to vesicles located on the oil/water interface.

The first composition may be in the form of a powder, or even a gel.

Second Cosmetic Composition

The second composition may be transparent, possibly colorless, and may be for applying to the lips, the nails, or the skin, for example. It may comprise at least one of the components described above for the first composition.

The second composition may include at least one coloring agent, e.g. one of those listed above.

Where appropriate, the second composition may present magnetic properties, but in many implementations of the invention, only the first composition presents magnetic properties.

The second composition may be for covering the first composition or for being covered by the first composition.

When the second composition is colored, its color may optionally contrast with the color of the first composition.

The second composition may have the same dosage form as the first, and the above description concerning the composition medium and the possible active ingredients also applies to the second composition.

Magnetic Devices

The magnetic device may comprise a permanent magnet or an electromagnet powered by at least one optionally-rechargeable battery, for example. For a battery, the magnetic device may include a switch enabling the electromagnet to be powered selectively with electricity.

The magnetic device may be arranged so as to create a magnetic field of orientation that varies over time. When the magnetic device comprises a magnet, the device may, for example, include a motor enabling the magnet to be rotated. In a variant, the magnetic device may comprise a plurality of solenoids disposed so as to generate a rotating magnetic field when powered sequentially with electricity.

By way of example, a rotating magnetic field may make it possible to obtain a pattern presenting circular symmetry, e.g. a pattern giving the impression of a sphere in relief.

The electromagnet(s) may be powered continuously or intermittently, as desired by the user. In particular, the magnetic device may be arranged so that the electromagnets(s) need not be powered while the magnetic device is not correctly positioned close to the surface coated with the first composition.

The magnetic field is at least 50 milli teslas (mT), for example, and preferably at least 66 mT.

In order to make it easier to apply the magnetic field, the magnetic device may include a member enabling it to be positioned relative to the surface on which the first composition has been deposited. This makes it possible to prevent the magnetic device from accidentally coming into contact with the composition and/or makes it possible to center the pattern formed on the region under consideration.

In an implementation of the invention, the magnetic device is secured to an applicator, e.g. the applicator that is used to apply the first cosmetic composition. This makes it possible to reduce the number of objects that need to be manipulated by the user and makes it easier to apply makeup.

In another implementation of the invention, the magnetic device comprises a magnet mounted at a first end of a rod having a second end that is connected to a handle of an applicator, e.g. the applicator that is used to apply the first cosmetic composition.

The magnetic field may also be exerted by means of a magnetic structure, in particular a flexible structure, including alternate N and S poles. By way of example, such a structure may make it possible to form repeated patterns, e.g. stripes, on the first composition.

A Method of Applying Makeup

According to another of its aspects, the invention also provides a method of applying makeup to a surface, which method comprises the following steps:

depositing at least first and second cosmetic compositions in the fluid state on the surface, the first composition covering or being covered by the second composition, the first composition containing magnetic particles that are movable under the effect of a magnetic field; and

exposing at least part of the first composition to a magnetic field, so as to orientate and/or displace at least a fraction of the magnetic particles, and so as to modify the appearance of the first composition.

Exposure to the magnetic field can take place before and/or after the second composition has been applied to the surface or to the first composition.

The magnetic field may be applied so as to form at least one pattern on the first composition, said pattern being linked to the shape of the field lines, for example.

As mentioned above, when the second cosmetic composition is applied to the first, it makes it possible to obtain a depth, gloss, smoothness, or other effect. The second composition may be transparent. The second composition may alternatively be covered by the first, so as to create a colored background/base, for example.

By way of example, the second composition may be for application to the lips or to the nails.

The magnetic field may be applied so as to model the clarity and/or the color of at least a region of the face or of the body to which the first composition has been applied.

For example, when the cosmetic composition is a foundation, orienting the particles under the effect of the magnetic field makes it possible to modify the clarity of the composition and thus to model the appearance of the face in the regions exposed to the magnetic field, in particular so as to apply makeup of cameo type. By way of example, the magnetic field may be applied so as to darken the sides of the face, so as to make it appear thinner than it really is.

The magnetic field may be applied until the first composition obtains a fixed appearance, i.e. the appearance of said composition ceases to vary even if the magnetic field continues to exist. In a variant, the magnetic field may be applied for a period of time that is shorter than the period of time that causes all of the magnetic particles in the exposed region to be permanently displaced and/or oriented.

Since the clarity and/or the color of the first composition change progressively under the effect of the magnetic field, the user can stop subjecting the magnetic particles to the field when the first composition presents the desired appearance.

In an implementation of the invention, the magnetic field is exerted through a magnetic sheet. Depending on the shape of said sheet, the field lines will have different shapes, thereby making it possible to increase the number of patterns that can be produced with a single magnet, for example.

The magnetic field may be exerted successively on different regions of the surface that are coated with the first composition.

The magnetic field may be exerted on regions of the surface that are disjoint, so as to create separate patterns, for example.

A region of the surface--coated with the first composition need not be exposed to the magnetic field, so as not to modify the appearance of the first composition in said region after it has been deposited.

The first and second compositions may be applied in various ways, e.g. by means of a cosmetics applicator that is preferably non-magnetic and that is selected from brushes, flocked endpieces, and foams, for example, or it may be applied without using an applicator, with the first and second compositions being spread on with the fingers or sprayed on, for example.

In an implementation of the invention, at least one of the first and second compositions is applied to the surface through a perforated mask. This makes it possible to produce a predetermined pattern corresponding to the shape of the perforation, for example. At least one region of the surface covered in the first composition may then be exposed to the magnetic field.

### BRIEF DESCRIPTION OF THE DRAWINGS

### EXAMPLES

The invention will be better understood on reading the following detailed description of non-limiting implementations thereof, and on examining the accompanying drawings, in which:

FIG. 1 is a diagram showing an example of a kit of the invention;

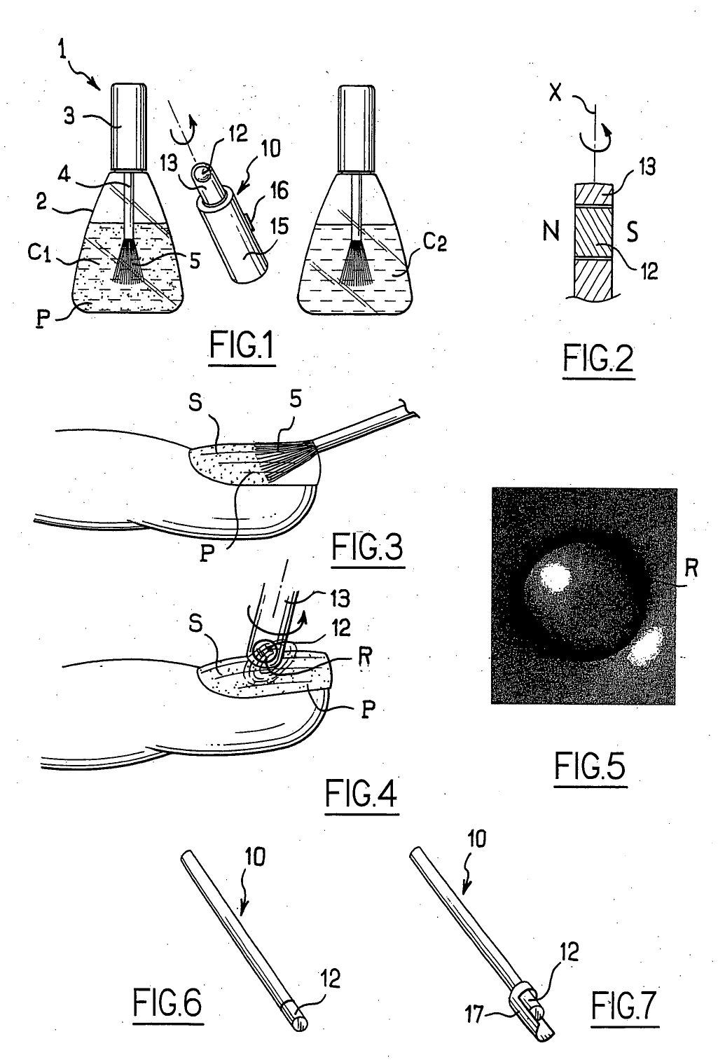

FIG. 2 is a diagrammatic and fragmentary axial section view showing the magnetic device of FIG. 1;

FIGS. 3 and 4 are diagrams showing the kit being used;

FIG. 5 shows an example of a pattern that can be obtained by means of the invention;

FIG. 6 is a diagram showing, in isolation, another example of a magnetic device that can be used;

FIG. 7 is a diagram showing the FIG. 6 magnetic device provided with a positioning member for positioning the magnet facing the made-up surface;

FIG. 8 is a diagram showing another example of a kit of the invention;

FIG. 9 is a diagram showing, in elevation and in isolation, an example of an applicator secured to a magnetic device;

FIG. 10 is a diagram showing another example of a magnetic device that can be used;

FIG. 11 shows another example of a packaging device for the first composition;

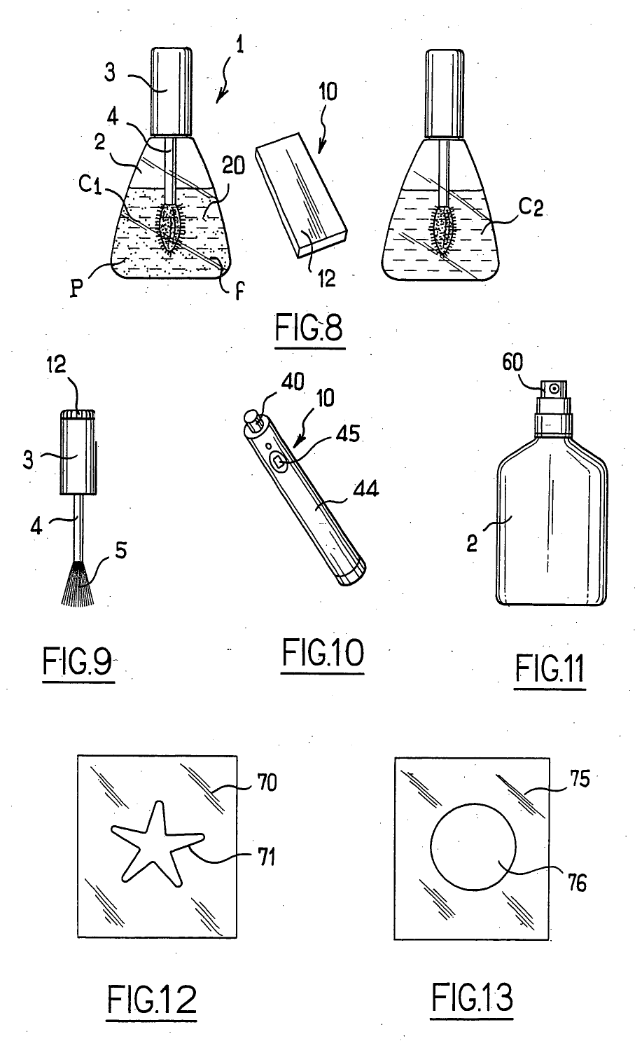

FIG. 12 shows a perforated mask that is suitable for being used during implementation of the method of the invention; and

FIG. 13 shows a magnetic sheet that is suitable for being used during implementation of the method of the invention.

### MORE DETAILED DESCRIPTION

In the figures, magnetic particles are shown in the form of dots in order to make the drawings easy to understand, but in reality the particles need not be visible to the naked eye.

FIG. 1 shows a kit 1 comprising a first cosmetic composition C₁ containing magnetic particles P having orientation and/or position that affects the appearance of the composition after it has been deposited on a surface such as the skin, the lips, the nails, hair, or even false nails.

The kit 1 also includes a second composition C₂, contained in a receptacle similar to that containing the first composition C₁, for example.

In the embodiment shown, the composition C1 is a nail varnish contained in a receptacle 2 that is closed by a cap 3. The cap supports a non-magnetic cosmetics applicator 4 including an applicator member 5 constituted by a brush enabling the varnish to be applied to the nails.

The kit 1 further comprises a magnetic device 10 that makes it possible to generate a magnetic field that is useful for changing the appearance of the first composition C1 without making contact therewith.

In the embodiment under consideration, the magnetic device 10 comprises a permanent magnet 12 supported by a support member 13 of longitudinal axis X, the polar axis of the magnet 12 being substantially perpendicular to the axis X.

In the embodiment under consideration, the magnetic device 10 is arranged to generate a rotating magnetic field, and includes a motor (not shown), housed in a casing 15, so as to rotate the support member 13 about it axis X.

A switch 16 is present on the casing 15 so as to enable the user to switch on the motor, thereby rotating the support member 13 together with the magnet 12.

In a variant not shown, the rotating magnetic field is generated by a plurality of solenoids that are powered sequentially so as to generate a rotating field.

In order to use the kit 1, the user can begin, as shown in FIG. 3, by applying the first composition C₁ by means of an applicator 4 to the surface S to be made up, specifically a fingernail.

In the subsequent step shown in FIG. 4, the user brings the magnetic device 10 over a central region R of the surface S and actuates the switch 16 so as to make the magnet 12 turn.

The magnetic particles contained in the first composition C₁ tend to come into alignment with the field lines of the magnet 12 and change orientation, thereby leading to a change in the appearance of the composition C₁.

The user can choose the length of time the magnetic field is to be applied as a function of the desired result.

By way of example, the pattern obtained can give the impression of a sphere in relief, as shown in FIG. 5.

The user then applies the second composition C₂, which is a transparent varnish, for example, once the first composition C₁ has dried.

Applying the second composition C₂ makes it possible to create an effect of additional depth, for example.

In the nail varnish embodiment in FIGS. 1 to 5, the first composition C₁ may have the following formulation, with quantities being expressed in percentages by weight in all of the examples below.

### Example A

First composition

| | |
|---|---|
| Nitrocellulose | 11 |
| N-ethyl o,p-toluenesulfonamide | 5 |
| Alkyde resin | 10 |
| Isopropanol | 4 |
| Magnetic pigments* | 0.5 |
| Butyl acetate/ethyl acetate 50/50 | Qsp 100 |

*Nacres containing at least 14% of Fe₃O4, referenced COLORONA PATINA GOLD (117288), and sold by MERCK.

The appearance of such a nail varnish can be changed by applying a magnetic field before the varnish has had time to dry.

A second composition C₂ having the following formulation, for example, may be applied to the first, after the first has dried.

Second composition

| | |
|---|---|
| Nitrocellulose | 11 |
| N-ethyl o,p-toluenesulfonamide | 5 |
| Alkyde resin | 10 |
| Isopropanol | 4 |
| Butyl acetate/ethyl acetate 50/50 | Qsp 100 |

In a variant embodiment of the invention, the second composition C₂ is applied before the first composition C₁, so as to create a colored background/base, for example.

The first composition C₁ can thus be less covering.

The following example is an example of a second composition for creating a colored background/base, the first composition having the formulation of above-mentioned example A, for example.

### Example B

Second composition

| | |
|---|---|
| Nitrocellulose | 11 |
| N-ethyl o,p-toluenesulfonamide | 4 |
| Alkyde resin | 6 |
| Isopropanol | 4 |
| DC RED7 CI 15850 pigments | 2 |
| Butyl acetate/ethyl acetate 50/50 | Qsp 100 |

The first composition in example A contains only one type of magnetic pigment.

The following example shows the possibility of having, within the first composition, magnetic pigments and another coloring substance, e.g. pigments having an optical effect, in this event goniochromatic pigments.

### Example C

First cosmetic composition

| | |
|---|---|
| Nitrocellulose | 11 |
| N-ethyl o,p-toluenesulfonamide | 5 |
| Alkyde resin | 10 |
| Isopropanol | 4 |
| COLORONA BLACKSTAR GOLD, MERCK ® (magnetic | 2.5 |
| pigments) | |
| SICOPEARL FANTASTICO ROSE, BASF ® | 2.5 |
| (goniochromatic pigments) | |
| Butyl acetate/ethyl acetate 50/50 | Qsp 100 |

The magnetic pigment is gold in color, and said color is present on the color path of the goniochromatic pigment.

Without magnetic excitation, the mixture presents a goniochromatic effect without any visible pattern, the color of the magnetic pigment making it possible to avoid masking the goniochromatic effect. It is possible to see a surface having a base color varying from gold to pink.

In contrast, after applying a magnetic field, the magnetic pigments coming into alignment with the field lines create a pattern that becomes superimposed on the color changes that result from the goniochromatic pigment. A gold-colored pattern obtained by means of the particles of oriented magnetic pigment can appear on a pink base for some orientation conditions concerning the observer and/or the made-up surface.

A second composition having, for example, the same formulation as that of Example A, may be applied to the first, after the first has dried.

The first composition may also contain magnetic pigments and diffractive pigments, for example.

### Example D

First composition

| | |
|---|---|
| Nitrocellulose | 11 |
| N-ethyl o,p-toluenesulfonamide | 5 |
| Alkyde resin | 10 |
| Isopropanol | 4 |
| Magnetic pigments* | 0.5 |
| Pigments having a diffractive optical effect** | 3 |
| Butyl acetate/ethyl acetate 50/50 | Qsp 100 |

*Nacres containing at least 14% of Fe₃O4, referenced COLORONA PATINA GOLD (117288), and sold by MERCK.
**Pigments sold under the trade name SPECTRAFLAIR by FLEX FLEXPRODUCTS.

By way of example, the second composition is that of Example A.

Naturally, whatever the nature of the first and second compositions, the magnetic field applied to the first need not rotate. By way of example, FIG. 6 shows a magnetic device which, at its end, includes a permanent magnet 12 in the form of a bar.

When the magnetic field does not rotate, the user can, for example, move the magnet into the vicinity of the first composition as a function of the desired result.

Whatever its nature, the magnetic device may include a member enabling it to be positioned relative to the surface S.

By way of example, the positioning member serves to prevent the magnetic device from touching the composition while the magnetic field is being exerted.

The positioning member can also serve to center the pattern that is produced relative to the surface S, e.g. the nail.

Depending on the nature of the surface, the positioning member could take various forms, e.g. that of an extension 17 offering an abutment surface for engaging the end of the finger, as shown in FIG. 7.

FIG. 8 shows another embodiment of a kit 1 of the invention, including a first composition C₁ and a second composition C₂, respectively constituted in this embodiment by a liquid lipstick and a lipgloss.

In this embodiment, the applicator 4 comprises a flocked endpiece 20 supported by the cap 3 of the receptacle 2.

By way of example, the magnetic device 10 is in the form of a flexible structure, e.g. made of plastics material filled with magnetized particles, creating alternate N and S poles, thereby making it possible to form repeated patterns, e.g. stripes, on the surface coated with the first composition.

By way of example, for lipstick, the first composition C₁ presents the following formulation.

### Example E

First composition

| | |
|---|---|
| Octyl-2 dodecanol | 10 |
| Ditertiobutyl 4-hydroxytoluene | 0.07 |
| Polybutene (monoolefins/isoparaffins 95/5) | 50 |
| (PM: 2060) | |
| A mixture of isopropyl, isobutyl, and n-butyl | 0.4 |
| p-hydroxybenzoates (40/30/30) | |
| Pentaerythrityl tetraisostearate | 11.33 |
| Tridecyle trimellitate | 13 |
| 2-decyl tetradecanoic acid triglyceride | 15 |
| (GUERBET C24) | |
| Magnetic pigments* | 0.2 |

*Nacres containing at least 14% of Fe₃O4, sold under the reference CLOISONNE NU ANTIQUE GREEN 828 CB by ENGELHARD.

By way of example, the second composition has the following formulation.

Second composition

| | |
|---|---|
| Degussa Aerosil R972 | 5 |
| Hydrogenated polyisobutylene (Parleam oil) | 2.1 |
| Octyldodecanol | 0.9 |
| Phenylated silicone oil (Dow Corning 556C) | 2.1 |
| Polyvinyl pyrrolidone and Eicosene copolymer | 1.2 |
| (ISP Anatron V220) | |
| Isododecane | Qsp 100 |

The second composition may be applied to the first and makes it possible to create an effect of depth.

Whatever the type of applicator, the magnet 12 may, where appropriate, be incorporated in the applicator.

In the embodiment in FIG. 9, the closure cap 3 is surmounted by the magnet 12 on the side remote from the applicator member 5.

In the embodiment in FIG. 13, the magnet 12 is supported by a support member 13 surmounted by a cap 51, and can, when not in use, be housed in a compartment 50 of the cap 3 for closing the receptacle 2 containing the first composition C₁. The cap 51 serves as a handle for the magnet 12, and also serves to close the compartment 50.

It is not beyond the ambit of the present invention for the magnetic field to be generated by an electromagnet instead of by a permanent magnet.

FIG. 10 shows a magnetic device 10 comprising an electromagnet 40 at one end of a casing 44 housing the power supply.

A switch 45 enables the electromagnet 40 to be switched on selectively by the user.

Various devices other than those described above for packaging and/or dispensing or applying the compositions C₁ and C₂ can be used.

By way of example, at least one of the compositions C₁ and C₂ can be deposited on the surface S without using an applicator, but in the form of a spray, e.g. by using a pump 60 as shown in FIG. 11. The spray can also be generated by means of an airbrush or by a pressurized receptacle, for example.

The devices for packaging and/or dispensing or applying the first and second compositions can differ from each other.

A perforated mask 70, as shown in FIG. 12 in which its perforation pattern 71 is in the shape of a star, can be interposed between the spray and the surface to be made up.

An optionally-perforated sheet 75 that is permeable to the magnetic field can be interposed between the magnet 12 or the electromagnet 40 and the surface S, so as to change the shape of the field lines and create novel effects, as shown in FIG. 13.

Naturally, the invention is not limited to the examples given above.

For example, the kit may include a plurality of magnets having various shapes, so as to create various patterns.

Throughout the description, including in the claims, the expression “comprising a” should be understood as being synonymous with “comprising at least one” unless specified to the contrary.

What is claimed is:

1. A kit for applying makeup to a surface such as the skin, the nails, hair, or the lips, said kit comprising:
a first cosmetic composition including metallic iron particles, in particular soft iron; and
a second cosmetic composition for covering or for being covered by the first composition.

2. A kit for applying makeup to a surface such as the skin, the nails, hair, or the lips, said kit comprising:
a first cosmetic composition including magnetic particles that are movable under the effect of a magnetic field;
a second cosmetic composition for covering or for being covered by the first composition;
a magnetic device for generating a magnetic field that makes it possible to displace and/or modify the orientation of all or some of the magnetic particles when the first composition is applied in the form of at least one layer to the surface.

3. A kit according to claim 1, wherein the second composition is for covering the first.

4. A kit according to claim 1, wherein the second composition is transparent.

5. A kit according to claim 1, wherein the second composition is for being covered by the first.

6. A kit according to claim 1, wherein the second composition is colored.

7. A kit according to claim 1, including a magnetic device for generating the magnetic field that makes it possible to modify the appearance of the first composition.

8. A kit according to claim 1, wherein the first composition is a nail varnish.

9. A kit according to claim 1, wherein the first composition is a lipstick.

10. A kit according to claim 2, wherein the magnetic particles comprise a magnetic material selected from the group constituted by: iron, nickel, cobalt, and alloys and oxides thereof, in particular Fe₃O₄.

11. A kit according to claim 1, wherein the magnetic particles are aspherical.

12. A kit according to claim 1, wherein the first composition contains at least one coloring and/or reflective substance selected from the following: organic and/or inorganic pigments, composite pigments, nacre pigments, goniochromatic pigments, reflective particles, diffractive pigments, thermochrome agents, photochrome agents, piezochrome agents, solvatochrome agents.

13. A kit according claim 2., wherein the magnetic device comprises a magnet.

14. A kit according claim 2, wherein the magnetic device comprises an electromagnet.

15. A kit according to claim 1, wherein the magnetic device includes a switch enabling the electromagnet to be powered selectively with electricity.

16. A kit according to claim 2, wherein the magnetic device is arranged to generate a rotating field, and in particular it comprises a magnet and a motor enabling the magnet to be rotated.

17. A kit according to claim 2, wherein the magnetic device includes a member enabling it to be positioned relative to the surface on which the first composition has been deposited.

18. A kit according claim 2, wherein the magnetic device is secured to an applicator.

19. A kit according to claim 2, wherein the magnetic device comprises a magnet mounted at a first end of a rod having a second end that is connected to a handle of an applicator.

20. A method of applying makeup to a surface, such as the skin, the nails, hair, or the lips, said method comprising the following steps:
depositing at least first and second cosmetic compositions in the fluid state on the surface, the first composition covering or being covered at least in part by the second, the first composition containing magnetic particles that are movable under the effect of a magnetic field; and
exposing at least part of the first composition to a magnetic field, so as to change the orientation of and/or displace at least a fraction of the magnetic particles.

21. A method according to claim 20, wherein the magnetic field is applied so as to form at least one pattern on the first composition.

22. A method according to claim 20, wherein the magnetic field is applied so as to model the clarity and/or the color of at least a region of the face or of the body on which the first composition has been applied.

23. A method according to claim 20, wherein the magnetic field is exerted by a permanent magnet.

24. A method according to claim 23, wherein the magnet is rotated.

25. A method according to claim 20, wherein the magnetic field is exerted by an electromagnet.

26. A method according to claim 25, wherein the electromagnet is powered by at least one optionally-rechargeable battery.

27. A method according to claim 25, wherein the electromagnet is switched on selectively by the user.

28. A method according to claim 20, wherein the magnetic field is applied until the first composition obtains a fixed appearance.

29. A method according to claim 20, wherein the magnetic field is applied for a period of time that is shorter than the period of time that causes all of the magnetic particles in the exposed region to be permanently displaced and/or oriented.

30. A method according to claim 20, wherein the magnetic field is exerted successively on different regions of the surface that are coated with the first composition.

31. A method according to claim 30, wherein the magnetic field is exerted on regions of the surface that are disjoint.

32. A method according to claim 20, wherein at least one region of the surface that is coated with the first composition is not exposed to the magnetic field.

33. A method according to claim 20, wherein the first composition is applied by means of a cosmetics applicator.

34. A method according to claim 33, wherein the applicator comprises a brush, a flocked endpiece, or a foam.

35. A method according to claim 33, wherein the applicator is non-magnetic.

36. A kit according to claim 20, wherein the magnetic particles comprise a magnetic material selected from the group constituted by: iron, nickel, cobalt, and alloys and oxides thereof.

37. A method according to claim 20, wherein the first composition contains magnetic particles and non-magnetic particles.

38. A method according to claim 20, wherein the first composition is applied to the surface through a perforated mask.

39. A method according to claim 20, wherein the magnetic field is applied through a sheet that is permeable to the magnetic field.

40. A method according to claim 20, wherein, after a given drying time, the first composition takes on a state that prevents the magnetic particles from changing their orientation under the effect of a magnetic field.

41. A method according to claim 20, wherein the second composition is applied to the first.

42. A method according to claim 20, wherein the first composition is applied to the second.

43. A method according to claim 20, wherein the second composition is colorless.

44. A method according to claim 20, wherein the second composition is colored.

45. A method according to claim 20, wherein the second composition is transparent.
